(19) United States
(12) Patent Application Publication
(10) Pub. No.: US 2024/0307457 A1
(43) Pub. Date: Sep. 19, 2024

(54) Composition and Method for Treating Diseases Thereof

(71) Applicant: Biospring Medical Co., Ltd., New Taipei City (TW)

(72) Inventor: MENG-SHIUE WU, New Taipei City (TW)

(73) Assignee: Biospring Medical Co., Ltd., New Taipei City, TW (TW)

(21) Appl. No.: 18/610,250

(22) Filed: Mar. 19, 2024

Related U.S. Application Data
(60) Provisional application No. 63/491,079, filed on Mar. 19, 2023.
(60) Provisional application No. 63/491,081, filed on Mar. 20, 2023.
(60) Provisional application No. 63/491,082, filed on Mar. 20, 2023.

(51) Int. Cl. A61K 35/50; C12N 5/00; C12N 5/073

(52) U.S. Cl. CPC A61K 35/50; C12N 5/0031; C12N 5/0605; C12N 2501/2306; C12N 2501/24; C12N 2501/25

(57) ABSTRACT

A composition and a method for treating diseases thereof are provided, wherein the composition comprises placental decidual mesenchymal stem cells, wherein the placental decidual mesenchymal stem cells overexpress decoy receptor 3 (DcR3) by stimulating with TNF-α, IFN-γ, IL-6 or their combination thereof, and wherein the diseases including neurological diseases, eye diseases, and lung diseases.

### CROSS-REFERENCE TO RELATED APPLICATION

This application claims the benefits of U.S. Provisional Patent Application No. 63/491,079 filed on Mar. 19, 2023, U.S. Provisional Patent Application No. 63/491,081 filed on Mar. 20, 2023, and U.S. Provisional Patent Application No. 63/491,082 filed on Mar. 20, 2023, which are incorporated by reference herein in its entirety.

### FIELD OF THE INVENTION

The present invention is related to a composition and a method for treating diseases thereof. Particularly, to a composition and a method for treating neurological diseases, eye diseases and lung diseases.

### BACKGROUND OF THE INVENTION

Nerve cell, as known as neuron, is composed of soma (cell body) and neurite (neuronal process), the basic component of neuron comprises axon, dendrites, myelin, and nucleus. Neurons are typically classified into three types based on their function: sensory neurons, motor neurons, and interneurons.

Neurological diseases are some of the most common medical conditions in the world, including neuroinflammation, Alzheimer's disease (AD), Parkinson's disease (PD), and Multiple Sclerosis (MS).

Eyes are an important organ that are responsible for receiving and processing visual detail, as well as enabling several photo response functions that are independent of vision, Eye diseases some of the most common medical conditions in the world, including dry eye, cataract, glaucoma, cornea inflammation, conjunctivitis and age-related macular degeneration (AMD).

Lung diseases are some of the most common medical conditions in the world, including pneumonia, chronic obstructive pulmonary disease (COPD), acute lung injury (ALI) and acute respiratory distress syndrome (ARDS).

### DETAILED DESCRIPTION OF THE INVENTION

The present invention is based on [symbol] [symbol] DcR3 [symbol] [symbol] [symbol] [symbol] ,

[symbol]

[symbol]

[symbol]

A composition for treating neurological diseases is provided in the present invention, comprising placental decidual mesenchymal stem cells (DMSCs), wherein the placental decidual mesenchymal stem cells overexpress decoy receptor 3 (DcR3) by stimulating with Tumor Necrosis Factor-α (TNF-α), Interferon-γ (IFN-γ), Interleukin-6 (IL-6) or their combination thereof.

A method for treatment of neurological diseases is also provided in the present invention, comprising administering a composition comprises placental decidual mesenchymal stem cells (DMSCs) to a subject in need thereof, wherein the placental decidual mesenchymal stem cells overexpress decoy receptor 3 (DcR3) by stimulating with Tumor Necrosis Factor-α (TNF-α), Interferon-γ (IFN-γ), Interleukin-6 (IL-6), or their combination thereof.

In the present invention, the neurological diseases preferably comprise neuroinflammation related disease or apoptosis of nerve cells.

In the present invention, the neurological diseases most preferably comprise stroke, trauma, Alzheimer's disease (AD), Parkinson's disease (PD), Multiple Sclerosis (MS), spinal cord injury, or peripheral nerve injury.

The present invention is based on [symbol] [symbol] DcR3 [symbol] [symbol] [symbol] [symbol] ,

[symbol]

[symbol]

[symbol]

[symbol]

A composition for treating eye diseases is provided in the present invention, comprising placental decidual mesenchymal stem cells, wherein the placental decidual mesenchymal stem cells (DMSCs) overexpress decoy receptor 3 (DcR3) by stimulating with Tumor Necrosis Factor-α (TNF-α), Interferon-γ (IFN-γ), Interleukin-6 (IL-6) or their combination thereof.

A method for treatment of eye diseases is also provided in the present invention, comprising administering a composition comprises placental decidual mesenchymal stem cells to a subject in need thereof, wherein the placental decidual mesenchymal stem cells (DMSCs) overexpress decoy receptor 3 (DcR3) by stimulating with Tumor Necrosis Factor-α (TNF-α), Interferon-γ (IFN-γ), Interleukin-6 (IL-6), or their combination thereof.

In the present invention, the eye diseases preferably comprise dry eye, cataract, glaucoma, cornea inflammation, conjunctivitis or age-related macular degeneration (AMD).

The present invention is based on [symbol] [symbol] DcR3 [symbol] [symbol] [symbol] [symbol] ,

[symbol]

[symbol]

[symbol]

A composition for treating lung diseases is provided in the present invention, comprising placental decidual mesenchymal stem cells (DMSCs), wherein the placental decidual mesenchymal stem cells overexpress decoy receptor 3 (DcR3) by stimulating with Tumor Necrosis Factor-α (TNF-α), Interferon-γ (IFN-γ), Interleukin-6 (IL-6) or their combination thereof.

A method for treatment of lung diseases is also provided in the present invention, comprising administering a composition comprises placental decidual mesenchymal stem cells (DMSCs) to a subject in need thereof, wherein the placental decidual mesenchymal stem cells overexpress decoy receptor 3 (DcR3) by stimulating with Tumor Necrosis Factor-α (TNF-α), Interferon-γ (IFN-γ), Interleukin-6 (IL-6), or their combination thereof.

In the present invention, the lung diseases preferably comprise pneumonia, chronic obstructive pulmonary disease (COPD), acute lung injury (ALI) or acute respiratory distress syndrome (ARDS).

In the present invention, the placental decidual mesenchymal stem cells (DMSCs) overexpress DcR3 is prepared by the following steps:
culturing the DMSCs from human in a culture dish containing a serum-free medium until the DMSCs from human adhere to the culture dish;
adding at least one inflammatory cytokine at an effective amount to the culture dish containing a serum-free medium, wherein the at least one inflammatory cytokine is Tumor Necrosis Factor-α (TNF-α), Interferon-γ (IFN-γ), Interleukin-6 (IL-6), or their combination thereof;
allowing the at least one inflammatory cytokine to stimulate expression of DcR3 in the DMSCs from human for 48 hours; and
obtaining the DMSCs from human having increased expression level of DcR3 compared to DMSCs from human cultured without the stimulation of the at least one inflammatory cytokine, wherein the increased in statistically significant with a P value of less than 0.05.

### BRIEF DESCRIPTION OF THE DRAWINGS

FIG. 1 shows the survival rate of mouse with the treatment of bone-marrow mesenchymal stem cells and placental decidual mesenchymal stem cells. The results indicate that [symbol] [symbol] , [symbol] 12%, [symbol] [symbol] [symbol] 38% ○

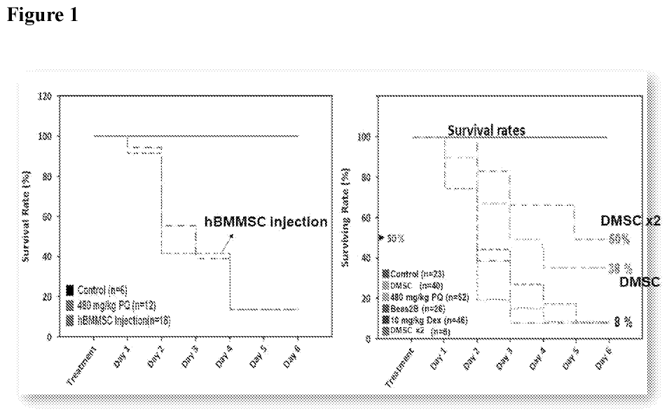

### EMBODIMENTS

To make the above-described and other purposes, features, and advantages of the present invention more obvious and easier to understand, preferred embodiments are provided below, and are described in detail as follows:

### EXAMPLE 1

### Methods

(1) [symbol] [symbol] ( [symbol] TNF-α, IFN-γ, IL-6) [symbol] 24˜72 [symbol] [symbol]
(2) [symbol] [symbol] ( [symbol] TNF-α, IFN-γ, IL-6) [symbol] , [symbol] [symbol] [symbol] , [symbol] [symbol] [symbol] , [symbol] [symbol] [symbol] [symbol] , [symbol] [symbol]
(3) [symbol] [symbol] [symbol] , [symbol] anti-DcR3 antibody [symbol] DcR3 [symbol] , [symbol] anti-DcR3 antibody [symbol] ,

[symbol]

[symbol]

[symbol]

[symbol]

### Results

[symbol]

[symbol]

[symbol]

[symbol]

[symbol] [symbol] , [symbol] [symbol] [symbol] [symbol] [symbol] [symbol] [symbol] [symbol] ,

[symbol]

[symbol]

[symbol] anti-DcR3 antibody [symbol] , [symbol] [symbol] [symbol] [symbol] [symbol] [symbol] [symbol] [symbol] ,

[symbol]

[symbol]

### Conclusion

[symbol] [symbol] , [symbol] [symbol] [symbol] [symbol] DcR3, [symbol] [symbol] [symbol] [symbol] [symbol] [symbol] ,

[symbol]

[symbol]

[symbol]

[symbol]

### EXAMPLE 2

### Methods

(1) [symbol] [symbol] ( [symbol] TNF-α, IFN-γ, IL-6) [symbol] 24˜72 [symbol]
(2) [symbol] [symbol] ( [symbol] TNF-α, IFN-γ, IL-6) [symbol] , [symbol] [symbol] [symbol] , [symbol] [symbol] [symbol] , [symbol] [symbol] [symbol] , [symbol] [symbol]
(3) [symbol] (2) [symbol] [symbol] , [symbol] anti-DcR3 antibody [symbol] DcR3 [symbol] , [symbol] anti-DcR3 antibody [symbol] ,

[symbol]

[symbol]

[symbol]

### Results

[symbol]

[symbol]

[symbol]

[symbol]

[symbol]

[symbol]

[symbol] [symbol] , [symbol] [symbol] [symbol] [symbol] [symbol] [symbol] [symbol] [symbol] ,

[symbol]

[symbol]

[symbol]

[symbol] anti-DcR3 antibody [symbol] , [symbol] [symbol] [symbol] [symbol] [symbol] [symbol] [symbol] [symbol] ,

[symbol]

[symbol]

### Conclusion

[symbol] [symbol] , [symbol] [symbol] [symbol] [symbol] DcR3, [symbol] [symbol] [symbol] [symbol] [symbol] [symbol] ,

[symbol]

[symbol]

[symbol]

[symbol]

[symbol]

### EXAMPLE 3

### Methods

(1) [symbol] [symbol] ( [symbol] TNF-α, IFN-γ, IL-6) [symbol] 24˜72 [symbol]
(2) [symbol] [symbol] ( [symbol] TNF-α, IFN-γ, IL-6) [symbol] , [symbol] [symbol] [symbol] , [symbol] [symbol] [symbol] , [symbol] [symbol] [symbol] , [symbol] [symbol]
(3) [symbol] (2) [symbol] [symbol] [symbol] , [symbol] anti-DcR3 antibody [symbol] DcR3 [symbol] , [symbol] anti-DcR3 antibody [symbol] , [symbol] [symbol] [symbol]
(4) [symbol] [symbol] , [symbol] DcR3 [symbol] [symbol] [symbol] DcR3

[symbol]

[symbol]

[symbol]

### Results

[symbol]

[symbol]

[symbol]

[symbol]

[symbol]

[symbol] [symbol] , [symbol] [symbol] [symbol] [symbol] [symbol] [symbol] ,

[symbol]

[symbol]

[symbol]

[symbol] anti-DcR3 antibody [symbol] , [symbol] [symbol] [symbol] [symbol] [symbol] [symbol] [symbol] [symbol] ,

[symbol]

[symbol]

[symbol] [symbol] [symbol] [symbol] [symbol] [symbol] , [symbol] [symbol] DcR3 [symbol] [symbol] [symbol] ,

[symbol]

[symbol]

### Conclusion

[symbol] [symbol] , [symbol] [symbol] [symbol] [symbol] DcR3, [symbol] [symbol] [symbol] ,

[symbol]

[symbol]

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The cells, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A composition for treating neurological diseases, comprising placental decidual mesenchymal stem cells (DMSCs), wherein the placental decidual mesenchymal stem cells (DMSCs) overexpress decoy receptor 3 (DcR3) by stimulating with Tumor Necrosis Factor-α (TNF-α), Interferon-γ (IFN-γ), Interleukin-6 (IL-6) or their combination thereof.

2. The composition of claim 1, wherein the placental decidual mesenchymal stem cells (DMSCs) overexpress DcR3 is prepared by the following steps:
culturing the DMSCs from human in a culture dish containing a serum-free medium until the DMSCs from human adhere to the culture dish;
adding at least one inflammatory cytokine at an effective amount to the culture dish containing a serum-free medium, wherein the at least one inflammatory cytokine is Tumor Necrosis Factor-α (TNF-α), Interferon-γ (IFN-γ), Interleukin-6 (IL-6), or their combination thereof;
allowing the at least one inflammatory cytokine to stimulate expression of DcR3 in the DMSCs from human for 48 hours; and
obtaining the DMSCs from human having increased expression level of DcR3 compared to DMSCs from human cultured without the stimulation of the at least one inflammatory cytokine, wherein the increased in statistically significant with a P value of less than 0.05.

3. The composition of claim 1, wherein the neurological diseases comprise neuroinflammation related disease or apoptosis of nerve cells.

4. The composition of claim 1, wherein the neurological diseases comprise stroke, trauma, Alzheimer's disease (AD), Parkinson's disease (PD), Multiple Sclerosis (MS), spinal cord injury, or peripheral nerve injury.

5. A method for treatment of neurological diseases, comprising administering a composition comprises placental decidual mesenchymal stem cells (DMSCs) to a subject in need thereof, wherein the placental decidual mesenchymal stem cells (DMSCs) overexpress decoy receptor 3 (DcR3) by stimulating with Tumor Necrosis Factor-α (TNF-α), Interferon-γ (IFN-γ), Interleukin-6 (IL-6), or their combination thereof.

6. The method of claim 5, wherein the placental decidual mesenchymal stem cells (DMSCs) overexpress DcR3 is prepared by the following steps:
culturing the DMSCs from human in a culture dish containing a serum-free medium until the DMSCs from human adhere to the culture dish;
adding at least one inflammatory cytokine at an effective amount to the culture dish containing a serum-free medium, wherein the at least one inflammatory cytokine is Tumor Necrosis Factor-α (TNF-α), Interferon-γ (IFN-γ), Interleukin-6 (IL-6), or their combination thereof;
allowing the at least one inflammatory cytokine to stimulate expression of DcR3 in the DMSCs from human for 48 hours; and
obtaining the DMSCs from human having increased expression level of DcR3 compared to DMSCs from human cultured without the stimulation of the at least one inflammatory cytokine, wherein the increased in statistically significant with a P value of less than 0.05.

7. The method of claim 5, wherein the neurological diseases comprise neuroinflammation related disease or apoptosis of nerve cells.

8. The method of claim 5, wherein the neurological diseases comprise stroke, trauma, Alzheimer's disease (AD), Parkinson's disease (PD), Multiple Sclerosis (MS), spinal cord injury, or peripheral nerve injury.

9. A composition for treating eye diseases, comprising placental decidual mesenchymal stem cells (DMSCs), wherein the placental decidual mesenchymal stem cells (DMSCs) overexpress decoy receptor 3 (DcR3) by stimulating with Tumor Necrosis Factor-α (TNF-α), Interferon-γ (IFN-γ), Interleukin-6 (IL-6), or their combination thereof.

10. The composition of claim 9, wherein the placental decidual mesenchymal stem cells (DMSCs) overexpress DcR3 is prepared by the following steps:
culturing the DMSCs from human in a culture dish containing a serum-free medium until the DMSCs from human adhere to the culture dish;
adding at least one inflammatory cytokine at an effective amount to the culture dish containing a serum-free medium, wherein the at least one inflammatory cytokine is Tumor Necrosis Factor-α (TNF-α), Interferon-γ (IFN-γ), Interleukin-6 (IL-6), or their combination thereof;
allowing the at least one inflammatory cytokine to stimulate expression of DcR3 in the DMSCs from human for 48 hours; and
obtaining the DMSCs from human having increased expression level of DcR3 compared to DMSCs from human cultured without the stimulation of the at least one inflammatory cytokine, wherein the increased in statistically significant with a P value of less than 0.05.

11. The composition of claim 9, wherein the eye diseases comprise dry eye, cataract, glaucoma, cornea inflammation, conjunctivitis or age-related macular degeneration (AMD).

12. A method for treatment of eye diseases, comprising administering a composition comprises placental decidual mesenchymal stem cells (DMSCs) to a subject in need thereof, wherein the placental decidual mesenchymal stem cells (DMSCs) overexpress decoy receptor 3 (DcR3) by stimulating with Tumor Necrosis Factor-α (TNF-α), Interferon-γ (IFN-γ), Interleukin-6 (IL-6), or their combination thereof.

13. The method of claim 12, wherein the placental decidual mesenchymal stem cells (DMSCs) overexpress DcR3 is prepared by the following steps:
culturing the DMSCs from human in a culture dish containing a serum-free medium until the DMSCs from human adhere to the culture dish;
adding at least one inflammatory cytokine at an effective amount to the culture dish containing a serum-free medium, wherein the at least one inflammatory cytokine is Tumor Necrosis Factor-α (TNF-α), Interferon-γ (IFN-γ), Interleukin-6 (IL-6), or their combination thereof;
allowing the at least one inflammatory cytokine to stimulate expression of DcR3 in the DMSCs from human for 48 hours; and
obtaining the DMSCs from human having increased expression level of DcR3 compared to DMSCs from human cultured without the stimulation of the at least one inflammatory cytokine, wherein the increased in statistically significant with a P value of less than 0.05.

14. The method of claim 12, wherein the eye diseases comprise dry eye, cataract, glaucoma, cornea inflammation, conjunctivitis or age-related macular degeneration (AMD).

15. A composition for treating lung diseases, comprising placental decidual mesenchymal stem cells (DMSCs), wherein the placental decidual mesenchymal stem cells (DMSCs) overexpress decoy receptor 3 (DcR3) by stimulating with Tumor Necrosis Factor-α (TNF-α), Interferon-γ (IFN-γ), Interleukin-6 (IL-6), or their combination thereof.

16. The composition of claim 15, wherein the placental decidual mesenchymal stem cells (DMSCs) overexpress DcR3 is prepared by the following steps:
culturing the DMSCs from human in a culture dish containing a serum-free medium until the DMSCs from human adhere to the culture dish;
adding at least one inflammatory cytokine at an effective amount to the culture dish containing a serum-free medium, wherein the at least one inflammatory cytokine is Tumor Necrosis Factor-α (TNF-α), Interferon-γ (IFN-γ), Interleukin-6 (IL-6), or their combination thereof;
allowing the at least one inflammatory cytokine to stimulate expression of DcR3 in the DMSCs from human for 48 hours; and
obtaining the DMSCs from human having increased expression level of DcR3 compared to DMSCs from human cultured without the stimulation of the at least one inflammatory cytokine, wherein the increased in statistically significant with a P value of less than 0.05.

17. The composition of claim 15, wherein the lung diseases comprise pneumonia, chronic obstructive pulmonary disease (COPD), acute lung injury (ALI) or acute respiratory distress syndrome (ARDS).

18. A method for treatment of lung diseases, comprising administering a composition comprises placental decidual mesenchymal stem cells (DMSCs) to a subject in need thereof, wherein the placental decidual mesenchymal stem cells (DMSCs) overexpress decoy receptor 3 (DcR3) by stimulating with Tumor Necrosis Factor-α (TNF-α), Interferon-γ (IFN-γ), Interleukin-6 (IL-6), or their combination thereof.

19. The method of claim 18, wherein the placental decidual mesenchymal stem cells (DMSCs) overexpress DcR3 is prepared by the following steps:
culturing the DMSCs from human in a culture dish containing a serum-free medium until the DMSCs from human adhere to the culture dish;
adding at least one inflammatory cytokine at an effective amount to the culture dish containing a serum-free medium, wherein the at least one inflammatory cytokine is Tumor Necrosis Factor-α (TNF-α), Interferon-γ (IFN-γ), Interleukin-6 (IL-6), or their combination thereof;
allowing the at least one inflammatory cytokine to stimulate expression of DcR3 in the DMSCs from human for 48 hours; and
obtaining the DMSCs from human having increased expression level of DcR3 compared to DMSCs from human cultured without the stimulation of the at least one inflammatory cytokine, wherein the increased in statistically significant with a P value of less than 0.05.

20. The composition of claim 18, wherein the lung diseases comprise pneumonia, chronic obstructive pulmonary disease (COPD), acute lung injury (ALI) or acute respiratory distress syndrome (ARDS).
